(19) United States
(12) Patent Application Publication
(10) Pub. No.: US 2015/0211045 A1
(43) Pub. Date: Jul. 30, 2015

(54) MICROFLUIDIC METHOD AND SYSTEM FOR ENZYME INHIBITION ACTIVITY SCREENING

(71) Applicant: Caliper Life Sciences, Inc., Hopkinton, MA (US)

(72) Inventors: Jude A. Dunne, Menlo Park, CA (US); Javier A. Farinas, Los Altos, CA (US); Irina G. Kazakova, Los Gatos, CA (US); Esther Huang, Mountain View, CA (US); Raphaele Gerber, Foster City, CA (US); Roger Dettloff, Emerald Hills, CA (US); Thi Ngoc Vy Trinh, Cupertino, CA (US); Coleen Hacker, Cupertino, CA (US)

(21) Appl. No.: 14/682,996

(22) Filed: Apr. 9, 2015

Related U.S. Application Data
(62) Division of application No. 11/899,599, filed on Sep. 5, 2007, now abandoned.
Application No. 11/899,599 is a continuation-in-part of application No. 11/051,445, filed on Feb. 3, 2005, now abandoned.
Application No. 11/051,445 is a continuation-in-part of application No. 09/993,314, filed on Nov. 5, 2001, now Pat. No. 7,105,304.
(60) Provisional application No. 60/246,617, filed on Nov. 7, 2000.
(60) Provisional application No. 60/845,655, filed on Sep. 18, 2006.

(51) Int. Cl. C12Q 1/48; B01L 3/00

(52) U.S. Cl. CPC C12Q 1/485; B01L 3/502715; B01L 3/50273; G01N 2500/20; G01N 2500/04; B01L 2300/0838; B01L 2300/14; B01L 2300/0829

(57) ABSTRACT

Methods for screening a compound for enzyme inhibition activity include providing at least one sample mixture to a microfluidic device, applying vacuum pressure to the sample mixture, flowing the sample mixture along a microchannel of the microfluidic device, separating at least two components of the sample mixture based upon a net charge difference between the product and at least one other material to produce separated material, detecting at least one of the separated materials, and determining enzyme inhibition activity based on the detection of the separated material. Kits for screening a compound for enzyme inhibition activity include a first multiwell plate having a specific plurality of enzymes disposed within a first plurality of wells and a second multiwell plate having a plurality of enzyme substrates disposed with a second plurality of wells, a phosphate source and a cofactor disposed within each well of the second plate.

### CROSS-REFERENCES TO RELATED APPLICATIONS

This application is a divisional of, and claims the benefit of, U.S. patent application Ser. No. 11/899,599 filed Sep. 5, 2007, which is a continuation-in-part of, and claims the benefit of, U.S. patent application Ser. No. 11/051,445 filed Feb. 3, 2005, which is a continuation-in-part of U.S. patent application Ser. No. 09/993,314 filed Nov. 5, 2001, now U.S. Pat. No. 7,105,304, which claims the benefit of and priority to U.S. Provisional Application No. 60/246,617 filed Nov. 7, 2000, the disclosures of which are herein incorporated by reference. This application also claims the benefit of U.S. Provisional Application No. 60/845,655 filed Sep. 18, 2006, the disclosure of which is herein incorporated by reference.

### FIELD OF THE INVENTION

The present invention relates to kits, systems, and methods for determining the level of enzyme activity in the presence of a compound, and more particularly to kits, systems, and methods for identifying drugs that modulate kinase activity across an array of kinases.

### COPYRIGHT NOTIFICATION

Pursuant to 37 C.F.R. §1.71(e), Applicants note that a portion of this disclosure contains material that is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Kinases are enzymes that catalyze the transfer of a phosphate group from ATP (adenosine triphosphate) or another nucleoside triphosphate to a substrate. For example, hexokinase catalyzes the phosphoryl transfer from ATP to glucose to produce glucose-6-phosphate as an initial step in glycolysis. Other kinases involved in glycolysis include phosphofructokinase, phosphoglycerate kinase, and pyruvate kinase. Kinases are also involved, e.g., in protein phosphorylation by transferring the terminal phosphate from ATP to a side chain of an amino acid residue of a protein. In eukaryotic cells, protein phosphorylation serves various functions including, e.g., the phosphorylation of cell-surface receptors to produce intracellular effects, the regulation of the cell cycle, the protection of cells from toxic changes in metabolites, or the like.

Protein kinases comprise a large family of enzymes with over 500 different forms identified to date in the human genome. Protein substrates for these enzymes may include up to one-third of all cellular proteins. An understanding of the protein kinase enzymes and their targets is crucial to understanding cellular regulation and cellular pathology.

Protein kinases play very important roles in many cell functions, including, but not limited to, cellular metabolism, signal transduction, transcriptional regulation, cell motility, cell division, cellular signaling processes, cellular proliferation, cellular differentiation, apoptosis, and secretion. These processes are mediated by phosphorylation or dephosphorylation of enzymes, protein substrates, transcription factors, hormone or growth factor receptors, and other cellular proteins. Protein phosphorylation is a regulatory mechanism used by cells to selectively modify proteins carrying regulatory signals from outside the cell to the nucleus.

Protein kinases are also involved in mediating the response to naturally occurring toxins and pathogens, which alter the phosphorylation states of proteins. Additionally, protein kinases are related to many epidemiologically relevant oncogenes and tumor suppressor genes. In fact, protein kinases are implicated in several hundred human diseases. Examples include neurodegenerative diseases such as amyotrophic lateral sclerosis and Alzheimer's disease. Consequently, protein kinases are often validated drug targets since human diseases are frequently linked to dysregulation of cellular signaling pathways.

With phosphorylation involved in so many cell functions and diseases, identifying compounds that affect protein kinase activity is tremendously important, not only to provide drug candidates that are active in treating disease, but also to ensure that these drug candidates act without causing adverse effects on other cellular functions. Thus, there is a need for high-throughput screening assays to identify activators and inhibitors of kinases as well as kits for carrying out such assays.

### SUMMARY OF THE INVENTION

One aspect of the present invention is a kit for screening a compound for enzyme inhibition activity. The kit includes first and second multiwell plates. The first multiwell plate has a plurality of enzymes disposed within a first plurality of wells. The second multiwell plate has a plurality of enzyme substrates disposed within a second plurality of wells. A phosphate source is disposed within each well of the second plate, the phosphate source disposed in the well at a predetermined concentration. In addition, a cofactor is disposed within each well of the second plate, the cofactor disposed in each well at a predetermined concentration.

Another aspect of the invention is a system for screening a compound for enzyme inhibition activity. The system includes first and second multiwell plates and a microfluidic device. The first multiwell plate includes a plurality of enzymes disposed within a first plurality of wells. The second multiwell plate includes a plurality of enzyme substrates disposed within a second plurality of wells. A phosphate source is disposed in each well of the second plate, the phosphate source disposed in the well at a predetermined concentration. A cofactor is also disposed in each well of the second plate, the cofactor disposed in each well at a predetermined concentration. The microfluidic device includes at least one microchannel and a capillary element, the capillary element operably connected to and in fluid communication with the microchannel. A detector is operably connected to the microfluidic device. The system further includes a computer. A controller and a fluid direction system are operably connected to the computer The fluid direction system includes a pressure source in fluid communication with the microfluidic device and a sample source, the sample source in fluid communication with the capillary element.

Yet another aspect of the present invention is a method of screening a compound for enzyme inhibition activity. A sample mixture comprising an enzyme, an enzyme substrate, a test compound, and a product is prepared using a kit including first and second multiwell plates, the first multiwell plate having a plurality of enzymes disposed within a first plurality of wells, the second multiwell plate having a plurality of enzyme substrates disposed within a second plurality of wells, wherein a phosphate source is disposed at a predetermined concentration within each well of the second plate, and wherein a cofactor is disposed at a predetermined concentration within each well of the second plate. The sample mixture is provided to a microfluidic device. Vacuum pressure is applied to the sample mixture, flowing the sample mixture along a microchannel of the microfluidic device. The sample mixture is separated based upon a difference in electrophoretic mobility between the product and the enzyme substrate. The separated product and enzyme substrate are detected, and enzyme inhibition activity is determined based on the detection of the separated product and enzyme substrate.

Many additional aspects of the invention will be apparent upon review, including uses of the devices and systems of the invention, methods of manufacture of the devices and systems of the invention, kits for practicing the methods of the invention, and the like. For example, kits comprising any of the devices or systems set forth above, or elements thereof, in conjunction with packaging materials (e.g., containers, sealable plastic bags, etc.) and instructions for using the devices, e.g., to practice the methods herein, are also contemplated.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof.

### BRIEF DESCRIPTION OF THE DRAWINGS

FIG. 1 schematically illustrates a system for screening a compound for enzyme inhibition activity, in accordance with the present invention;

FIG. 2 illustrates one embodiment of an enzyme plate used in accordance with the present invention;

FIG. 3 is a table illustrating components and reaction conditions for one embodiment of a screening kit, in accordance with the present invention;

FIG. 4 is a table illustrating components and reaction conditions for another embodiment of a screening kit, in accordance with the present invention;

FIG. 5 schematically illustrates a portion of a microfluidic device such as is illustrated in FIG. 1, in accordance with the present invention;

FIG. 6 illustrates enzyme degradation data for ABL;

FIG. 7 illustrates the change in conversion per week for a subset of enzymes that was tested for degradation over 22 weeks (error bars=95% confidence intervals);

FIG. 8 illustrates selectivity data for staurosporine;

FIG. 9 illustrates selectivity data for K252A; and

FIG. 10 illustrates selectivity data SB203580.

The drawings are not to scale.

### DETAILED DESCRIPTION

Before describing the present invention in detail, it is to be understood that this invention is not limited to particular compositions, devices, or systems, which can, of course, vary. It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which the invention pertains.

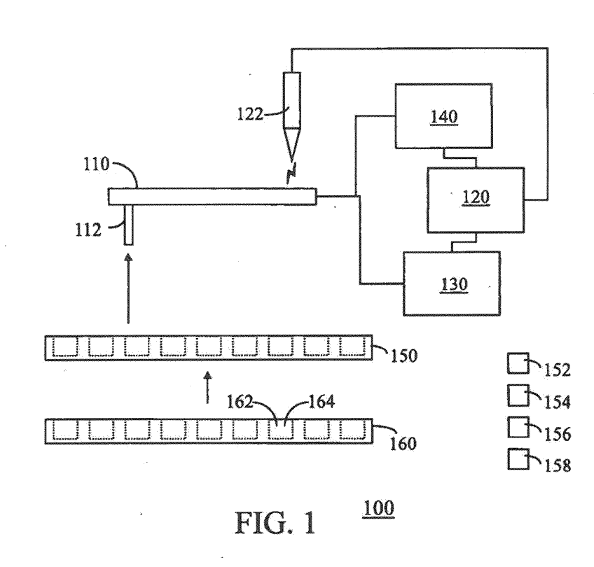

One aspect of the present invention is a kit for screening a compound for enzyme inhibition activity. In one embodiment, the kit includes first and second multiwell plates, i.e., enzyme plate 150 and substrate plate 160, a reconstitution buffer 152, DTT (dithiothreitol) 154, a protease inhibitor 156, and a termination buffer 158, all of which components are included in the system illustrated at 100 in FIG. 1. A plurality of enzymes are disposed within the wells of enzyme plate 150, and a plurality of enzyme substrates, a phosphate source 162, and a cofactor 164 are disposed within the wells of substrate plate 160.

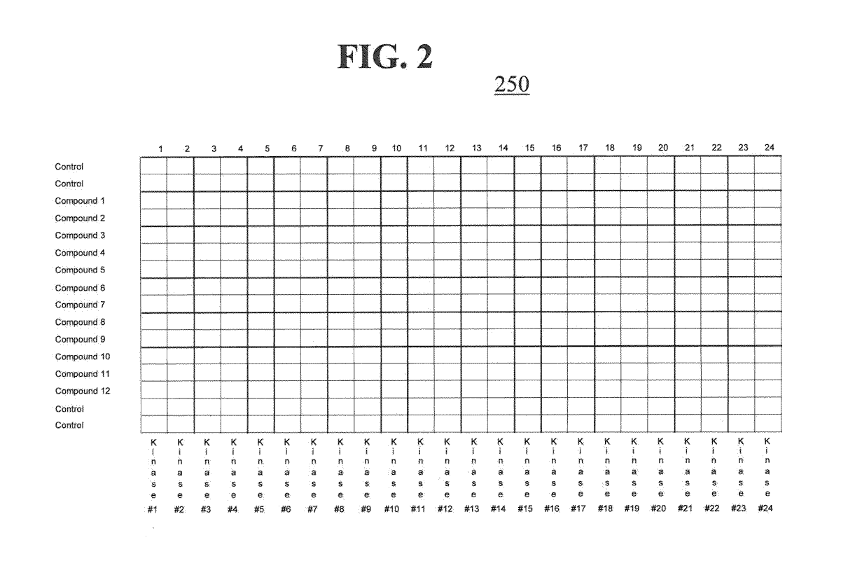

In one embodiment, enzyme plate 150 is an enzyme plate such as is illustrated in FIG. 2 at 250. Enzyme plate 250 includes a plurality of enzymes disposed within individual wells of plate 250. The arrangement of the enzymes on the enzyme plate is determined by the positioning of the corresponding substrates on the substrate plate. Positioning of the substrates is based on factors such as, for example, electrophoretic mobility.

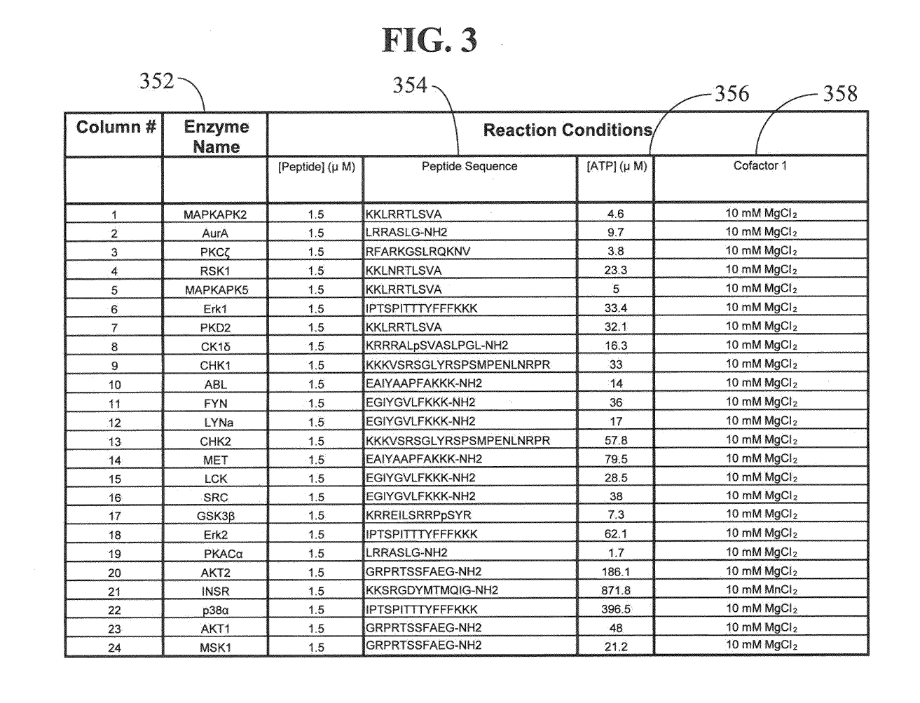

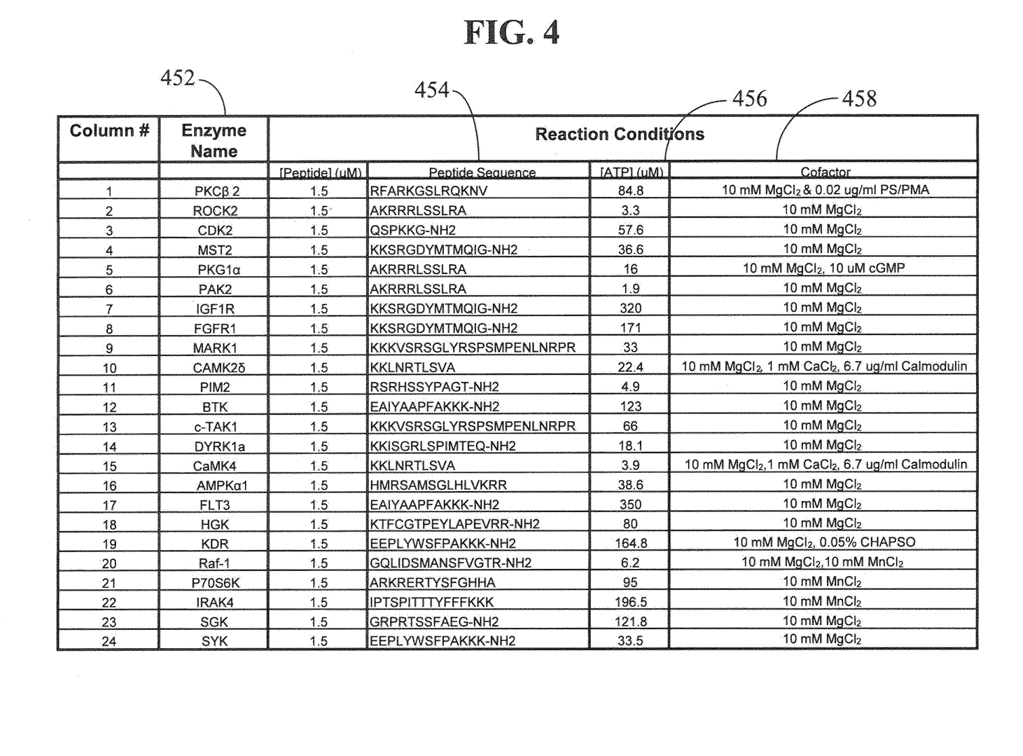

In this first illustrative embodiment, enzyme plate 250 includes a plurality of kinases numbered 1 through 24 disposed in columns 1 through 24 of plate 250. Kinases 1 through 24 comprise enzymes 352 illustrated in FIG. 3. Enzymes 352 include kinases MAPKAPK2, AurA, PKCζ, RSK1, MAPKAPK5, Erk1, PKD2, CK1δ, CHK1, ABL, FYN, LYNa, CHK2, MET, LCK, SRC, GSK3β, Erk2, PKACα, AKT2, INSR, p38α, AKT1, and MSK1. In this first embodiment, enzymes 352 are disposed within columns 1 through 24 in the exact order illustrated in FIG. 3. In a second illustrative embodiment, kinases 1 through 24 comprise enzymes 452 illustrated in FIG. 4. Enzymes 452 include kinases PKCβ2, ROCK2, CDK2, MST2, PKG1α, PAK2, IGF1R, FGFR1, MARK1, CAMK2δ, PIM2, BTK, c-TAK1, DYRK1a, CaMK4, AMPKα1, FLT3, HGK, KDR, Raf-1, P70S6K, IRAK4, SGK, and SYK. In this second embodiment, enzymes 452 are disposed within columns 1 through 24 in the exact order illustrated in FIG. 4. While 24 columns of kinases are illustrated in FIGS. 1-3, this is not intended to limit the invention. Other enzymes may be used, and the number of columns may be other than 24. The enzyme plate may also include a plurality of control wells as determined by the particular application.

Substrate plate 160 includes a plurality of enzyme substrates to be added to the enzyme plate. Each well of substrate plate 160 includes a solution containing a peptide, ATP (adenosine 5′-triphosphate), and a cofactor. Each enzyme acts on a specific peptide sequence. FIGS. 3 and 4 illustrate the peptide sequences 354, 454 for each corresponding enzyme 352, 452, respectively. FIGS. 3 and 4 also illustrate the specific concentration of ATP 356, 456 and the specific cofactor and cofactor concentration 358, 458 required for each enzyme 352, 452.

Kits in accordance with the present invention typically include all components necessary for screening a compound for enzyme inhibition activity, with the exception, of course, of the compound(s) of interest to be tested. For example, the kit may contain controls as indicated in FIG. 2, a reconstitution buffer 152, DTT 154 for addition to the reconstitution buffer prior to addition of the buffer to the enzyme plate, a protease inhibitor 156 also for addition to the reconstitution buffer prior to addition of the buffer to the enzyme plate, and a termination buffer 158 that is added to the enzyme plate to terminate the enzyme reaction prior to reading of the plate.

Essentially any kinase is suitable for use in the kits of the present invention, including those comprising EC (Enzyme Commission) numbers such as 2.1, 2.7, 2.8, 3.1, 3.4, 4.1, 6.2, or the like. These include, for example, a protein kinase, a protein kinase A, a protein kinase B, a protein kinase C, a hexokinase, a phosphofructokinase, a phosphoglycerate kinase, a pyruvate kinase, a cyclic AMP-dependent protein kinase, a cyclic GMP-dependent protein kinase, a calmodulin-dependent protein kinase II, a casein kinase I, a casein kinase II, a glycogen synthase kinase-3, a cyclin-dependent kinase (e.g., CDK2, CDK4, CDK6, or the like), a p34/cdc2 kinase, a nucleic acid kinase, or the like. FIGS. 3 and 4 provide examples of two kinase panels particularly suitable for use in the present invention.

In addition, the present invention may be adapted to other enzymes such as, for example phosphatases, proteases, and histone deacetylases (HDACs). In those embodiments, the materials to be separated include, e.g., a phosphatase enzyme substrate and product, a dephosphorylated product, or the like. Essentially any phosphatase is suitable for use in the assays of the present invention, just one example being those comprising the EC number 3.1.3. To further illustrate, phosphatase enzymes that are optionally used in the assays described herein include, e.g., a protein phosphatase, an acid phosphatase, an alkaline phosphatase, a sugar phosphatase, a polynucleotide phosphatase, or the like.

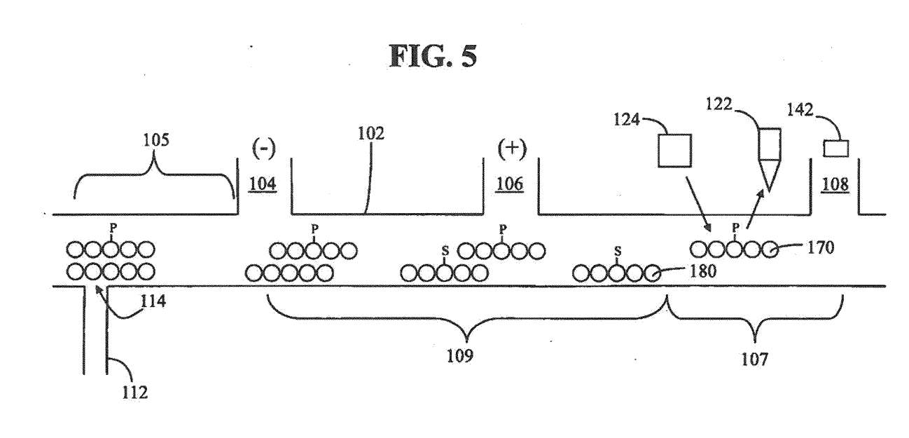

Another aspect of the invention is a system for screening a compound for enzyme inhibition activity. FIGS. 1 and 5, which are not to scale, illustrate one embodiment of a system 100 for screening compounds for enzyme activity, e.g., protein kinase activity, in accordance with the present invention. Note that the system is also suitable for use with other enzymes such as, for example, phosphatases, proteases, and histone deacetylases (HDACs).

System 100 includes a microfluidic device 110, a computer 120, a detector 122, a controller 130, and a fluid direction system 140. The system additionally includes a kit such as has been described above, including first and second multiwell plates 150 and 160, reconstitution buffer 152, DTT 154, protease inhibitor 156, and termination buffer 158.

Microfluidic device 110 includes at least one microchannel 102 and a plurality of reservoirs or ports 104, 106, 108 in fluid communication with microchannel 102. Aliquots of a sample containing a test compound (prepared in first and second multiwell plates 150 and 160) are flowed into main microchannel 102 from capillary element 112 towards reservoir 108 by, for example, applying a vacuum at reservoir 108 (or another point in the system) and/or by applying appropriate voltage gradients. As used herein, a “capillary element” includes an elongated body structure having a channel (e.g., a microchannel) disposed therethrough. A capillary element is preferably an integral extension of the body structure of a single microfluidic device, as illustrated in FIG. 1, but may also be a separate component that is temporarily coupled to one or more microfluidic device body structures. A microfluidic device in accordance with the present invention may include a single capillary element as illustrated in FIG. 1 or may include a plurality of capillary elements (e.g., 4 or 12).

Additional materials, such as buffer solutions, substrate solutions, enzyme solutions, and the like, are optionally flowed from reservoirs 104 or 106 and into main microchannel 102. Flow from these reservoirs is optionally performed by modulating fluid pressure, by electrokinetic approaches, or both (as illustrated in FIG. 5). A number of possible arrangements of channels within microfluidic device 110 are possible, the arrangement depending on the specific application. Channels within the microfluidic device are covered, as detailed below under the heading “Microfluidic Devices.”

Detector 122 is in sensory communication with main microchannel 102, detecting signals resulting, for example, from labeled materials flowing through the detection region 107. Detector 122 is optionally in sensory communication with any of the channels or regions of the device where detection is desired. As used herein, the phrase “in sensory communication” refers to positioning of a detector such that it is operably connected to the channel, i.e., capable of receiving a detectable signal from the contents of a channel. In the case of optical signals, this requires only that the detector be positioned to receive the optical signal. Detector 122 is also operably connected to computer 120, which digitizes, stores, and manipulates signal information detected by detector 122, for example, using any instruction set for determining enzyme activity, concentration, molecular weight or identity, or the like.

Fluid direction system 140 controls pressure, voltage, or both, e.g., at the reservoirs of the system or through the channels of the system, or at vacuum couplings fluidly coupled to main microchannel 102 or other channels described above. Optionally, as depicted, computer 120 controls fluid direction system 140. In one set of embodiments, computer 120 uses signal information to select further parameters for the microfluidic system. In certain embodiments, controller 130 dispenses aliquots of buffers or other materials into, for example, main microchannel 102. In these embodiments, controller 130 is also operably connected to computer 120, which directs controller 130 function.

Although not shown, a microfluidic device handler is also typically included in the integrated systems of the present invention. Microfluidic device handlers generally control, e.g., the x-y-z translation of microfluidic device 110 relative to multiwell plate 150 or other system components, under the direction of computer 120, to which the device handlers are operably connected. Microfluidic device handlers may also be involved in the transfer of materials from multiwell plate 160 to multiwell plate 150. Microfluidic devices, computers, detectors, controllers, and fluid flow systems in accordance with the present invention are described in more detail below.

Yet another aspect of the present invention provides a method of screening a compound for enzyme inhibition activity. A sample mixture is obtained using a kit such as has been described above, the sample mixture comprising an enzyme, an enzyme substrate, a test compound and a product. The sample mixture is provided to a microfluidic device such as has been described above. Vacuum pressure is applied to the sample mixture, flowing the sample mixture along a microchannel of the microfluidic device, thereby separating the sample mixture to produce separated materials. The separated materials are detected, and enzyme inhibition activity is determined based on the detection of the separated materials.

The compounds to be screened are prepared using a multiwell compound plate in addition to enzyme and substrate plates such as those illustrated at 150 and 160, respectively. The enzyme and substrate plates are stored frozen and are thawed prior to use.

Prepared compounds are added to enzyme plate 150 from the compound plate and incubated for a predetermined length of time as determined by the specific application. Reconstitution buffer 152, combined with DTT 154 and a protease inhibitor 156, is added to the enzyme plate either before adding the prepared compounds or in combination with the compounds. After expiration of the predetermined length of time, a portion of a substrate solution corresponding with a specific enzyme is removed from a well of substrate plate 160 and added to the corresponding well of enzyme plate 150. This mixture is left to incubate for a predetermined length of time. After expiration of this time, termination buffer 158 is added to each well of enzyme plate 150. See Example 1, below, for detailed steps. Aliquots of sample from each well are then provided to microfluidic device 110 for separation and analysis.

Each sample is flowed into region 105 of microchannel 102 via capillary element 112 by applying negative fluid pressure to the materials via a vacuum source applied to port 108. The materials are flowed into a microchannel separation region 109. Under an applied voltage, phosphorylated product is separated from unreacted substrate based upon a difference in mobilities of the phosphorylated product and the unreacted substrate. The separation region preferably contains free solution but may include an ion-exchange material (e.g., a polyarginine, a polylysine, a modified polyacrylamide, a modified dimethylacrylamide, or the like). Optionally, the ion-exchange material includes a polyacrylamide or a dimethylacrylamide modified (e.g., via covalent attachment, adsorption, or the like) by one or more anionic or cationic additives. In certain embodiments, the ion-exchange material is covalently or otherwise attached to a plurality of microbeads or a gel. In addition, the methods optionally include flowing the ion-exchange material into the separation region. The methods include detecting the resulting separated product and the unreacted substrate.

The sample is flowed through microchannel 102 and past detector 122. As the sample nears detector 122, a beam of light from LED (or laser or other light source) 124 is directed towards the sample. Fluorescence from the sample is then detected by detector 122. Signals from detector 122 are transmitted to computer 120 for analysis and determination of the level of enzyme inhibition activity, if any, caused by the compound of interest.

In certain embodiments, more than one product is formed by contacting the first and second materials. These are also optionally separated according to the methods described herein. The methods optionally include flowing materials in the microfluidic device in the absence of an applied electric field, or flowing materials in the microfluidic device under a simultaneously applied electric field.

FIG. 5 schematically illustrates one embodiment of an ion-exchange-induced mobility-shift-based separation method that is used for high-throughput compound screening in accordance with the present invention. As shown, microchannel configuration 100 includes microchannel 102 into which a sample including enzyme and substrate solutions is placed via capillary element 112. Under negative pressure applied by a vacuum 142 at vacuum port 108, the sample is continuously flowed from sample port 114 of main microchannel 102 towards port 108 and past detector 122 to detect a reaction product 170 and unreacted substrate 180. Optionally, a modulator, an inhibitor, an antagonist, an agonist, or other material may be flowed from a well on a multiwell plate or another external source through capillary element 112 into region 105 of main microchannel 102, e.g., to assess its impact on the assay.

A free solution or a solution of a suitable ion exchanger or other chromatographic material, as appropriate, is optionally placed into microchannel 102 and flowed continuously into separation region 109 of main microchannel 102 during the course of the assay to effect separation of the reaction product from unreacted substrate based upon their, e.g., respective mobilities or other distinguishing properties. Alternatively, separation region 109 of main microchannel 102 is selectively modified by, for example, coating it with the ion exchanger prior to commencing the assay. An additional option includes manufacturing at least separation region 109 of main microchannel 102 to possess the desired ion-exchange characteristics (e.g., by selecting appropriate microfluidic device substrate materials).

The method of screening a compound for enzyme inhibition activity includes mixing an enzyme solution (e.g., a protein kinase, a protein kinase A, a nucleic acid kinase, or the like) and a substrate solution to produce a phosphorylated product such as product 170 illustrated in FIG. 5.

### Example 1

The following example serves to illustrate, but not to limit, the present invention.

### Materials

A DeskTop Profiler™ instrument (available from Caliper Life Sciences, Inc.); ProfilerProm′ enzyme and substrate plates (available from Caliper Life Sciences, Inc.); a 384-well plate for control/compound preparation; reconstitution buffer, ATP/peptides, termination buffer, DTT, protease inhibitor, and DMSO; and microfluidic devices for performing mobility shift assays were used. The 384-well ProfilerProm′ enzyme plates include 24 enzymes per plate, pre-dispensed in columns of 16. The 384-well ProfilerPro™ substrate plates include 24 peptide/ATP pairs per plate, pre-dispensed in columns of 16. Prior to use, the plates are stored below −70° C. Buffers are stored below 4° C. DTT and protease inhibitor are stored below −70° C.

### Procedure

### 1. Prepare Components.

a. Remove reconstitution buffer and termination buffer from freezer and thaw. If stored for extended periods prior to use, maintain at 4° C.
b. To 50 mL reconstitution buffer, add 50 μL of 1M DTT and 500 μL of 100× protease inhibitor.
c. Prepare compounds.
   i. For 1 μL compound transfer, dilute compounds to 26× assay concentration in 100% DMSO; place in compound plate in rows C through N (see FIG. 2).
      OR
   ii. For 16 μL compound transfer, dilute compounds to 1.625× assay concentration in previously prepared reconstitution buffer with DTT and protease inhibitor; place in compound plate in rows C through N (see FIG. 2).
d. Prepare controls.
   i. For 1 μL control transfer, add 100% DMSO to compound plate in rows A, B, O, and P (see FIG. 2).
      OR
   ii. For 16 μL control transfer, make 6.25% DMSO solution in previously prepared reconstitution buffer with DTT and protease inhibitor; place in compound plate in rows A, B, 0, and P (see FIG. 2).
e. Place reconstitution buffer, compound plate, and termination buffer in incubator at 28° C.
   2. Prepare ProfilerPro™ substrate and enzyme plates.
a. Remove substrate plate from freezer and incubate at 28° C.
b. Wait 30 minutes
c. Remove enzyme plate from freezer and incubate at 28° C.; after 15 minutes, spin enzyme plate at 1000 rpm for 1 minute if bubbles are observed; remove seal.
d. Transfer compound/control from the compound plate to the enzyme plate.
   i. For 1 μL compound/control transfer, first add 15 μL of previously prepared reconstitution buffer with DTT and protease inhibitor to each well of the enzyme plate and mix; then add 1 μL compound/control from each well of the compound plate to a separate well of the enzyme plate; mix.
      OR
   ii. For 16 μL compound/control transfer, add 16 μL of the previously prepared mixture of compound/control and reconstitution buffer (with DTT and protease inhibitor) to each well of the enzyme plate and mix.
e. Pre-incubate the enzyme plate for up to 15 minutes at 28° C.
f. At least 60 minutes after placing the substrate plate in the incubator in Step a, remove the substrate plate from the incubator and spin at 500 rpm for 1 minute if bubbles are observed; remove seal.
g. Transfer 10 μL of substrate from each well of the substrate plate to each well of the enzyme plate; spin at 1000 rpm for 1 minute if bubbles are observed.
h. Cover enzyme plate and incubate for 90 minutes at 28° C.
i. Add 45 μL of termination buffer to each well of the enzyme plate in the same order used when adding substrate (to avoid variable incubation time); mix if necessary; spin at 1000 rpm for 1 minute.
   3. Read the enzyme plate using the DeskTop Profiler™ instrument.
   All assays are run at ATP/Km=1 for each enzyme. Common buffers are used:

### Reaction Buffer

100 mM HEPES, pH=7.5

10 mM MgCl₂ or MnCl₂

0.003% Brij-35

1 mM DTT

4% DMSO

[E] is predetermined to yield the appropriate conversion upon incubation and termination.

### Separation Buffer

100 mM HEPES, pH=7.5

0.015% Brij-35

1 mM EDTA

0.1% Coating Reagent 3

5% DMSO

### Results

### Enzyme Degradation.

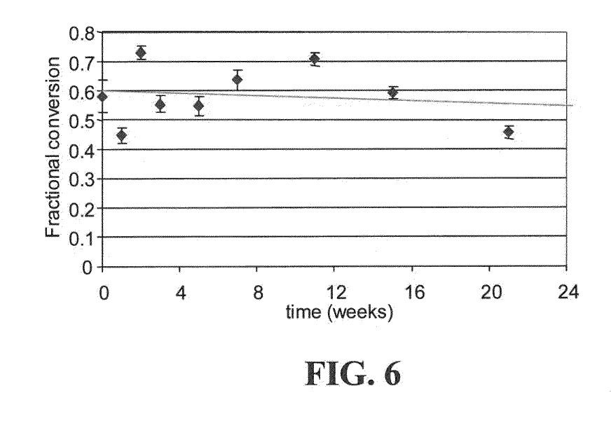

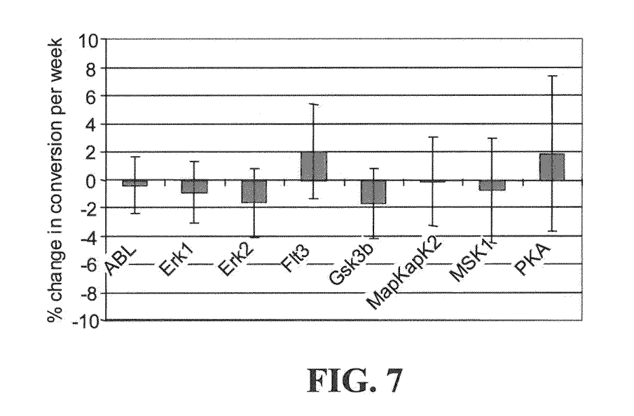

Enzymes were diluted to 260× the assay concentration in a storage buffer and 100 nL dispensed into the wells of a 384-well microtiter plate. Plates were frozen and stored at −80° C. Enzymes were assayed over 22 weeks by thawing and adding 16 μL of reconstitution buffer and 10 μL of substrate. After a 1-hour incubation, the reactions were quenched and run on a Caliper Life Sciences, Inc., LabChip® 3000 Drug Discovery System. Sample data are shown for ABL in FIG. 6. FIG. 7 shows the change in conversion per week for a subset of the enzymes that was tested for 22 weeks (error bars=95% confidence intervals). No enzyme showed a statistically significant difference from no change in activity.

### Kinase Selectivity Screening.

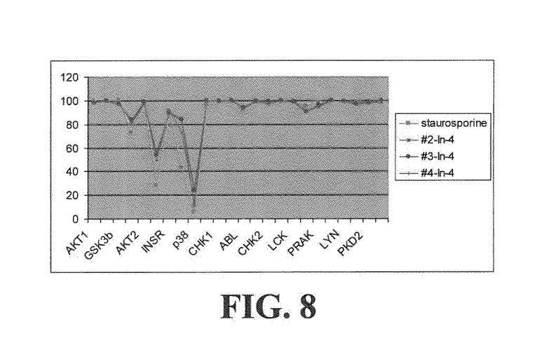

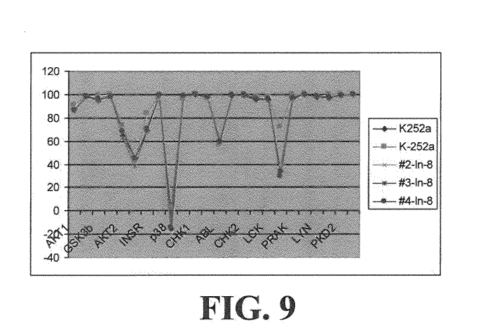

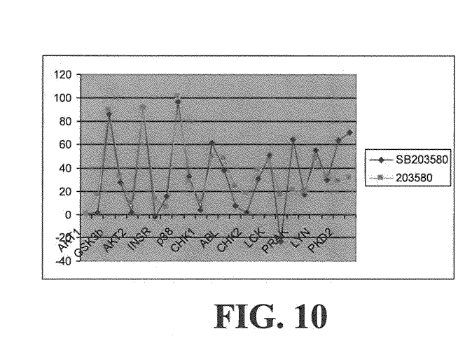

A set of 13 commercially available kinase inhibitors was run at a concentration of 10 μM against a ProfilerProm′ enzyme plate. In this example, the enzymes were disposed within columns 1 through 24 in the order of AKT1, MSK1, GSK3β, Erk1, AKT2, CK1δ, INSR, Erk2, p38, MET, CHK1, SRC, ABL, RSK1, CHK2, FYN, LCK, PKC, PRAK, AurA, LYN, MAPKAPK2, PKD2, PKA. Selectivity data for staurosporine (FIG. 8), K252A (FIG. 9), and SB203580 (FIG. 10) were run at Caliper Life Sciences, Inc. and at an external site, showing good agreement.

### Reproducibility.

Two ProfilerProm′ plates from two batches were run each of three days with DMSO controls in 5 wells. An ANOVA revealed an overall CV of 18.8% with the following sources of variability (% of total):

| | |
|---|---|
| day-to-day | 13.9% |
| batch-to-batch | 0% |
| plate-to plate | 26.5% |
| assay | 59.6% |

### Microfluidic Devices

Many different microscale systems are optionally adapted for use in the methods of the present invention. These systems are described in numerous publications by the inventors and their coworkers, including certain issued U.S. Patents, such as U.S. Pat. No. 5,699,157 (J. Wallace Parce) issued Dec. 16, 1997, U.S. Pat. No. 5,779,868 (J. Wallace Parce et al.) issued Jul. 14, 1998, U.S. Pat. No. 5,800,690 (Calvin Y. H. Chow et al.) issued Sep. 1, 1998, U.S. Pat. No. 5,842,787 (Anne R. Kopf-Sill et al.) issued Dec. 1, 1998, U.S. Pat. No. 5,852,495 (J. Wallace Parce) issued Dec. 22, 1998, U.S. Pat. No. 5,869,004 (J. Wallace Parce et al.) issued Feb. 9, 1999, U.S. Pat. No. 5,876,675 (Colin B. Kennedy) issued Mar. 2, 1999, U.S. Pat. No. 5,880,071 (J. Wallace Parce et al.) issued Mar. 9, 1999, U.S. Pat. No. 5,882,465 (Richard J. McReynolds) issued Mar. 16, 1999, U.S. Pat. No. 5,885,470 (J. Wallace Parce et al.) issued Mar. 23, 1999, U.S. Pat. No. 5,942,443 (J. Wallace Parce et al.) issued Aug. 24, 1999, U.S. Pat. No. 5,948,227 (Robert S. Dubrow) issued Sep. 7, 1999, U.S. Pat. No. 5,955,028 (Calvin Y. H. Chow) issued Sep. 21, 1999, U.S. Pat. No. 5,957,579 (Anne R. Kopf-Sill et al.) issued Sep. 28, 1999, U.S. Pat. No. 5,958,203 (J. Wallace Parce et al.) issued Sep. 28, 1999, U.S. Pat. No. 5,958,694 (Theo T. Nikiforov) issued Sep. 28, 1999, U.S. Pat. No. 5,959,291 (Morten J. Jensen) issued Sep. 28, 1999, U.S. Pat. No. 5,964,995 (Theo T. Nikiforov et al.) issued Oct. 12, 1999, U.S. Pat. No. 5,965,001 (Calvin Y. H. Chow et al.) issued Oct. 12, 1999, U.S. Pat. No. 5,965,410 (Calvin Y. H. Chow et al.) issued Oct. 12, 1999, U.S. Pat. No. 5,972,187 (J. Wallace Parce et al.) issued Oct. 26, 1999, U.S. Pat. No. 5,976,336 (Robert S. Dubrow et al.) issued Nov. 2, 1999, U.S. Pat. No. 5,989,402 (Calvin Y. H. Chow et al.) issued Nov. 23, 1999, U.S. Pat. No. 6,001,231 (Anne R. Kopf-Sill) issued Dec. 14, 1999, U.S. Pat. No. 6,011,252 (Morten J. Jensen) issued Jan. 4, 2000, U.S. Pat. No. 6,012,902 (J. Wallace Parce) issued Jan. 11, 2000, U.S. Pat. No. 6,042,709 (J. Wallace Parce et al.) issued Mar. 28, 2000, U.S. Pat. No. 6,042,710 (Robert S. Dubrow) issued Mar. 28, 2000, U.S. Pat. No. 6,046,056 (J. Wallace Parce et al.) issued Apr. 4, 2000, U.S. Pat. No. 6,048,498 (Colin B. Kennedy) issued Apr. 11, 2000, U.S. Pat. No. 6,068,752 (Robert S. Dubrow et al.) issued May 30, 2000, U.S. Pat. No. 6,071,478 (Calvin Y. H. Chow) issued Jun. 6, 2000, U.S. Pat. No. 6,074,725 (Colin B. Kennedy) issued Jun. 13, 2000, U.S. Pat. No. 6,080,295 (J. Wallace Parce et al.) issued Jun. 27, 2000, U.S. Pat. No. 6,086,740 (Colin B. Kennedy) issued Jul. 11, 2000, U.S. Pat. No. 6,086,825 (Steven A. Sundberg et al.) issued Jul. 11, 2000, U.S. Pat. No. 6,090,251 (Steven A. Sundberg et al.) issued Jul. 18, 2000, U.S. Pat. No. 6,100,541 (Robert Nagle et al.) issued Aug. 8, 2000, U.S. Pat. No. 6,107,044 (Theo T. Nikiforov) issued Aug. 22, 2000, U.S. Pat. No. 6,123,798 (Khushroo Gandhi et al.) issued Sep. 26, 2000, U.S. Pat. No. 6,129,826 (Theo T. Nikiforov et al.) issued Oct. 10, 2000, U.S. Pat. No. 6,132,685 (Joseph E. Kersco et al.) issued Oct. 17, 2000, U.S. Pat. No. 6,148,508 (Jeffrey A. Wolk) issued Nov. 21, 2000, U.S. Pat. No. 6,149,787 (Andrea W. Chow et al.) issued Nov. 21, 2000, U.S. Pat. No. 6,149,870 (J. Wallace Parce et al.) issued Nov. 21, 2000, U.S. Pat. No. 6,150,119 (Anne R. Kopf-Sill et al.) issued Nov. 21, 2000, U.S. Pat. No. 6,150,180 (J. Wallace Parce et al.) issued Nov. 21, 2000, U.S. Pat. No. 6,153,073 (Robert S. Dubrow et al.) issued Nov. 28, 2000, U.S. Pat. No. 6,156,181 (J. Wallace Parce et al.) issued Dec. 5, 2000, U.S. Pat. No. 6,167,910 (Calvin Y. H. Chow) issued Jan. 2, 2001, U.S. Pat. No. 6,171,067 (J. Wallace Parce) issued Jan. 9, 2001, U.S. Pat. No. 6,171,850 (Robert Nagle et al.) issued Jan. 9, 2001, U.S. Pat. No. 6,172,353 (Morten J. Jensen) issued Jan. 9, 2001, U.S. Pat. No. 6,174,675 (Calvin Y. H. Chow et al.) issued Jan. 16, 2001, U.S. Pat. No. 6,182,733 (Richard J. McReynolds) issued Feb. 6, 2001, U.S. Pat. No. 6,186,660 (Anne R. Kopf-Sill et al.) issued Feb. 13, 2001, U.S. Pat. No. 6,221,226 (Anne R. Kopf-Sill) issued Apr. 24, 2001, U.S. Pat. No. 6,233,048 (J. Wallace Parce) issued May 15, 2001, U.S. Pat. No. 6,235,175 (Robert S. Dubrow et al.) issued May 22, 2001, U.S. Pat. No. 6,235,471 (Michael Knapp et al.) issued May 22, 2001, U.S. Pat. No. 6,238,538 (J. Wallace Parce et al.) issued May 29, 2001, U.S. Pat. No. 6,251,343 (Robert S. Dubrow et al.) issued Jun. 26, 2001, U.S. Pat. No. 6,267,858 (J. Wallace Parce et al.) issued Jul. 31, 2001, U.S. Pat. No. 6,274,089 (Andrea W. Chow et al.) issued Aug. 14, 2001, U.S. Pat. No. 6,274,337 (J. Wallace Parce et al.) issued Aug. 14, 2001, U.S. Pat. No. 6,287,520 (J. Wallace Parce et al.) issued Sep. 11, 2001, U.S. Pat. No. 6,287,774 (Theo T. Nikiforov) issued Sep. 11, 2001, U.S. Pat. No. 6,303,343 (Anne R. Kopf-Sill) issued Oct. 16, 2001, U.S. Pat. No. 6,306,590 (Tammy Burd Mehta et al.) issued Oct. 23, 2001, and U.S. Pat. No. 6,306,659 (J. Wallace Parce et al.) issued Oct. 23, 2001.

Systems that are optionally adapted for use in the methods of the present invention are also described in, e.g., various published PCT applications, including WO 98/00231, WO 98/00705, WO 98/00707, WO 98/02728, WO 98/05424, WO 98/22811, WO 98/45481, WO 98/45929, WO 98/46438, and WO 98/49548, WO 98/55852, WO 98/56505, WO 98/56956, WO 99/00649, WO 99/10735, WO 99/12016, WO 99/16162, WO 99/19056, WO 99/19516, WO 99/29497, WO 99/31495, WO 99/34205, WO 99/43432, WO 99/44217, WO 99/56954, WO 99/64836, WO 99/64840, WO 99/64848, WO 99/67639, WO 00/07026, WO 00/09753, WO 00/10015, WO 00/21666, WO 00/22424, WO 00/26657, WO 00/42212, WO 00/43766, WO 00/45172, WO 00/46594, WO 00/50172, WO 00/50642, WO 00/58719, WO 00/60108, WO 00/70080, WO 00/70353, WO 00/72016, WO 00/73799, WO 00/78454, WO 01/02850, WO 01/14865, WO 01/17797, and WO 01/27253.

The methods of the invention are generally performed within fluidic channels. In some cases, as mentioned above, the channels are simply present in a capillary or pipettor element, e.g., a glass, fused silica, quartz, or plastic capillary. The capillary element is fluidly coupled to a source of, e.g., the reagent, sample, modulator, or other solution (e.g., by dipping the capillary element into a well on a microtiter plate), which is then flowed along the channel (e.g., a microchannel) of the element. The term “microfluidic,” as used herein, generally refers to one or more fluid passages, chambers or conduits which have at least one internal cross-sectional dimension, e.g., depth, width, length, diameter, etc., that is less than 1 mm, and typically between about 0.1 μm and about 500 μm.

In the devices of the present invention, the microscale channels or cavities typically have at least one cross-sectional dimension between about 0.1 μm and 200 μm, preferably between about 0.1 μm and 100 μm, and often between about 0.1 μm and 50 μm. Accordingly, the microfluidic devices or systems prepared in accordance with the present invention typically include at least one microscale channel, usually at least two intersecting microscale channels, and often, three or more intersecting channels disposed within a single body structure that make up a channel network. Channel intersections may exist in a number of formats, including cross intersections, “Y” and/or “T” intersections, or any number of other structures whereby two channels are in fluid communication.

The body structures of the microfluidic devices described herein are typically manufactured from two or more separate portions or substrates which when appropriately mated or joined together, form the microfluidic device of the invention, e.g., containing the channels and/or chambers described herein. During body structure fabrication, the microfluidic devices described herein will typically include a top portion, a bottom portion, and an interior portion, wherein the interior portion substantially defines the covered channels and chambers of the device.

In one aspect, a bottom portion of the unfinished device includes a solid substrate that is substantially planar in structure, and which has at least one substantially flat upper surface. Channels are typically fabricated on one surface of the device and sealed (i.e., covered) by overlaying the channels with an upper substrate layer. A variety of substrate materials are optionally employed as the upper or bottom portion of the device. Typically, because the devices are microfabricated, substrate materials will be selected based upon their compatibility with known microfabrication techniques, e.g., photolithography, wet chemical etching, laser ablation, air abrasion techniques, LIGA, reactive ion etching, injection molding, embossing, and other techniques. The substrate materials are also generally selected for their compatibility with the full range of conditions to which the microfluidic devices may be exposed, including extremes of pH, temperature, electrolyte concentration, and/or for their chromatographic properties. Accordingly, in some preferred aspects, the substrate material may include materials normally associated with the semiconductor industry in which such microfabrication techniques are regularly employed, including, e.g., silica-based substrates, such as glass, quartz, silicon or polysilicon, as well as other substrate materials, such as gallium arsenide and the like. In the case of semiconductive materials, it will often be desirable to provide an insulating coating or layer, e.g., silicon oxide, over the substrate material, and particularly in those applications where electric fields are to be applied to the device or its contents.

In additional preferred aspects, the substrate materials will comprise polymeric materials, e.g., plastics, such as polymethylmethacrylate (PMMA), polycarbonate, polytetrafluoroethylene (TEFLON™), polyvinylchloride (PVC), polydimethylsiloxane (PDMS), polysulfone, polystyrene, polymethylpentene, polypropylene, polyethylene, polyvinylidine fluoride, ABS (acrylonitrile-butadiene-styrene copolymer), and the like. In preferred embodiments, at least the separation region(s) is/are fabricated from polyacrylamide, dimethylacrylamide, modified versions thereof, nonionic detergents, ionic detergents, or the like. Such polymeric substrates are readily manufactured using available microfabrication techniques, as described above, or from microfabricated masters, using known molding techniques, such as injection molding, embossing or stamping, or by polymerizing the polymeric precursor material within the mold (see, e.g., U.S. Pat. No. 5,512,131). Such polymeric substrate materials are preferred for their ease of manufacture, low cost and disposability, as well as their general inertness to most extreme reaction conditions. Again, these polymeric materials optionally include treated surfaces, e.g., derivatized or coated surfaces, to enhance their utility in the microfluidic system, e.g., to provide enhanced fluid direction, e.g., as described in U.S. Pat. No. 5,885,470 (J. Wallace Parce et al.) issued Mar. 23, 1999, and which is incorporated herein by reference in its entirety for all purposes.

The channels and/or cavities of the microfluidic devices are typically fabricated into the upper surface of the bottom substrate or portion of the device, as microscale grooves or indentations, using the above described microfabrication techniques. The top portion or substrate also comprises a first planar surface, and a second surface opposite the first planar surface. In the microfluidic devices prepared in accordance with certain aspects of the methods described herein, the top portion can include at least one aperture, hole, or port disposed therethrough, e.g., from the first planar surface to the second surface opposite the first planar surface. In other embodiments, the port(s) is/are optionally omitted, e.g., where fluids are introduced solely through external capillary elements.

The first planar surface of the top portion or substrate is then mated, e.g., placed into contact with, and bonded to the planar surface of the bottom substrate, covering and sealing the grooves and/or indentations in the surface of the bottom substrate, to form the channels and/or chambers (i.e., the interior portion) of the device at the interface of these two components. Bonding of substrates is typically carried out by any of a number of different methods, e.g., thermal bonding, solvent bonding, ultrasonic welding, and the like. The finished body structure of a device is a unitary structure that houses, e.g., the channels and/or chambers of the device.

The hole(s) in the top of the finished device is/are oriented to fluidly communicate with at least one of the channels and/or cavities. In the completed device, the hole(s) optionally function as reservoirs for facilitating fluid or material introduction into the channels or chambers of the device, as well as providing ports at which, e.g., pressure elements (e.g., vacuum sources, etc.) are optionally placed into contact with fluids within the device, allowing application of pressure gradients along the channels of the device to control and direct fluid transport within the device. In optional embodiments, extensions are provided over these reservoirs to allow for increased fluid volumes, permitting longer running assays, and better controlling fluid flow parameters, e.g., hydrostatic pressures. Examples of methods and apparatus for providing such extensions are described in U.S. Pat. No. 6,251,343, filed Feb. 24, 1998, and incorporated herein by reference. These devices are optionally coupled to a sample introduction port, e.g., a pipettor or capillary element, which serially introduces multiple samples, e.g., from the wells of a microtiter plate. Thus, in some embodiments, both reservoirs in the upper surface and external capillary elements are present in a single device.

The sources of reagents, enzymes, substrates, samples, eluents, separation buffers, and other materials are optionally fluidly coupled to the microchannels in any of a variety of ways. In particular, those systems comprising sources of materials set forth in Knapp et al. “Closed Loop Biochemical Analyzers” (WO 98/45481; PCT/US98/06723) and U.S. Pat. No. 5,942,443 issued Aug. 24, 1999, entitled “High Throughput Screening Assay Systems in Microscale Fluidic Devices” to J. Wallace Parce et al. and, e.g., in 60/128,643 filed Apr. 4, 1999, entitled “Manipulation of Microparticles In Microfluidic Systems,” by Mehta et al. are applicable.

In these systems and as noted above, a capillary or pipettor element (i.e., an element in which components are optionally moved from a source to a microscale element such as a second channel or reservoir) is temporarily or permanently coupled to a source of material. The source is optionally internal or external to a microfluidic device that includes the pipettor or capillary element. Example sources include microwell plates, membranes or other solid substrates comprising lyophilized components, wells or reservoirs in the body of the microscale device itself and others.

### Flow of Materials in Microfluidic Systems

A preferred method of flowing materials along the microchannels or other cavities of the devices described herein is by pressure-based flow. Pressure is applied with or without a simultaneously applied electric field. Application of a pressure differential along a channel is carried out by any of a number of approaches. For example, it may be desirable to provide relatively precise control of the flow rate of materials, e.g., to precisely control incubation or separation times, etc. As such, in many preferred aspects, flow systems that are more active than hydrostatic pressure driven systems are employed. In certain cases, reagents may be flowed by applying a pressure differential across the length of the analysis channel. For example, a pressure source (positive or negative) is applied at the reagent reservoir at one end of the analysis channel, and the applied pressure forces the reagents/samples through the channel. The pressure source is optionally pneumatic, e.g., a pressurized gas, or a positive displacement mechanism, i.e., a plunger fitted into a reagent reservoir, for forcing the reagents through the analysis channel. Alternatively, a vacuum source is applied to a reservoir at the opposite end of the channel to draw the reagents through the channel. See, FIGS. 1 and 5. Pressure or vacuum sources may be supplied external to the device or system, e.g., external vacuum or pressure pumps sealably fitted to the inlet or outlet of the analysis channel, or they may be internal to the device, e.g., microfabricated pumps integrated into the device and operably linked to the analysis channel. Examples of microfabricated pumps have been widely described in the art. See, e.g., published International Application No. WO 97/02357.

In an alternative simple passive aspect, the reagents are deposited in a reservoir or well at one end of an analysis channel and at a sufficient volume or depth, that the reagent sample creates a hydrostatic pressure differential along the length of the analysis channel, e.g., by virtue of it having greater depth than a reservoir at an opposite terminus of the channel. The hydrostatic pressure then causes the reagents to flow along the length of the channel. Typically, the reservoir volume is quite large in comparison to the volume or flow through rate of the channel, e.g., 10 μl reservoirs, vs. 1000 μm² channel cross-section. As such, over the time course of the assay/separation, the flow rate of the reagents will remain substantially constant, as the volume of the reservoir, and thus, the hydrostatic pressure changes very slowly. Applied pressure is then readily varied to yield different reagent flow rates through the channel. In screening applications, varying the flow rate of the reagents is optionally used to vary the incubation time of the reagents. In particular, by slowing the flow rate along the channel, one can effectively lengthen the amount of time between introduction of reagents and detection of a particular effect. Alternatively, analysis channel lengths, detection points, or reagent introduction points are varied in fabrication of the devices, to vary incubation times. See also, “Multiport Pressure Control System,” by Chien and Parce, U.S. Ser. No. 60/184,390, filed Feb. 23, 2000, which describes multiport pressure controllers that couple pumps to multiple device reservoirs.

In further alternate aspects, hydrostatic, wicking and capillary forces are additionally, or alternately, used to provide for fluid flow. See, e.g., “Method and Apparatus for Continuous Liquid Flow in Microscale Channels Using Pressure Injection, Wicking and Electrokinetic Injection,” by Alajoki et al., U.S. Ser. No. 09/245,627, filed Feb. 5, 1999. In these methods, an adsorbent material or branched capillary structure is placed in fluidic contact with a region where pressure is applied, thereby causing fluid to move towards the adsorbent material or branched capillary structure.

In alternative aspects, flow of reagents is driven by inertial forces. In particular, the analysis channel is optionally disposed in a substrate that has the conformation of a rotor, with the analysis channel extending radially outward from the center of the rotor. The reagents are deposited in a reservoir that is located at the interior portion of the rotor and is fluidly connected to the channel. During rotation of the rotor, the centripetal force on the reagents forces the reagents through the analysis channel, outward toward the edge of the rotor. Multiple analysis channels are optionally provided in the rotor to perform multiple different analyses. Detection of a detectable signal produced by the reagents is then carried out by placing a detector under the spinning rotor and detecting the signal as the analysis channel passes over the detector. Examples of rotor systems have been previously described for performing a number of different assay types. See, e.g., Published International Application No. WO 95/02189. Test compound reservoirs are optionally provided in the rotor, in fluid communication with the analysis channel, such that the rotation of the rotor also forces the test compounds into the analysis channel.

For purposes of illustration, the discussion has focused on a single channel and accessing capillary; however, it will be readily appreciated that these aspects may be provided as multiple parallel analysis channels (e.g., each including mixing and separation regions) and accessing capillaries, in order to substantially increase the throughput of the system. Specifically, single body structures may be provided with multiple parallel analysis channels coupled to multiple sample accessing capillaries that are positioned to sample multiple samples at a time from sample libraries, e.g., multiwell plates. As such, these capillaries are generally spaced at regular distances that correspond with the spacing of wells in multiwell plates, e.g., 9 mm centers for 96 well plates, 4.5 mm for 384 well plates, and 2.25 mm for 1536 well plates.

In alternate aspects, an applied pressure is accompanied by the simultaneous application of an electric field to further effect fluid transport, e.g., through the mixing and/or separation regions of the microchannel. The electrokinetic transport systems of the invention typically utilize electric fields applied along the length of microchannels that have a surface potential or charge associated therewith. When fluid is introduced into the microchannel, the charged groups on the inner surface of the microchannel ionize, creating locally concentrated levels of ions near the fluid surface interface. Under an electric field, this charged sheath migrates toward the cathode or anode (depending upon whether the sheath comprises positive or negative ions) and pulls the encompassed fluid along with it, resulting in bulk fluid flow. This flow of fluid is generally termed electroosmotic flow. Where the fluid includes reagents (e.g., materials to be separated), the reagents are also pulled along. A more detailed description of controlled electrokinetic material transport systems in microfluidic systems is described in published International Patent Application No. WO 96/04547, which is incorporated herein by reference.

### Integrated Systems

The present invention, in addition to other integrated system components, also optionally includes a microfluidic device handler for performing the methods disclosed herein. Specifically, the microfluidic device handler includes a holder configured to receive the microfluidic device, a container sampling region proximal to the holder, and the controller. During operation of the handler, the controller directs, e.g., dipping of microfluidic device capillary or pipettor element(s) into a portion of, e.g., a microwell plate in the container sampling region. The microfluidic device handler also optionally includes a computer or a computer readable medium operably connected to the controller. The computer or the computer readable medium typically includes an instruction set for varying or selecting a rate or a mode of dipping capillary element(s) into fluid materials.

Although the devices and systems specifically illustrated herein are generally described in terms of the performance of a few or one particular operation, it will be readily appreciated from this disclosure that the flexibility of these systems permits easy integration of additional operations into these devices. For example, the devices and systems described will optionally include structures, reagents and systems for performing virtually any number of operations in addition to the operations specifically described herein. Aside from fluid handling, assays, and separation of sample and/or reaction components, other upstream or downstream operations include, e.g., extraction, purification, amplification, cellular activation, labeling reactions, dilution, aliquotting, labeling of components, assays and detection operations, electrokinetic or pressure-based injection of components or materials into contact with one another, or the like. Assay and detection operations include, without limitation, cell fluorescence assays, cell activity assays, receptor/ligand assays, immunoassays, or the like.

In the present invention, the separated materials are optionally monitored and/or detected so that, e.g., an activity can be determined. The systems described herein generally include microfluidic device handlers, as described above, in conjunction with additional instrumentation for controlling fluid transport, flow rate and direction within the devices, detection instrumentation for detecting or sensing results of the operations performed by the system, processors, e.g., computers, for instructing the controlling instrumentation in accordance with preprogrammed instructions, receiving data from the detection instrumentation, and for analyzing, storing and interpreting the data, and providing the data and interpretations in a readily accessible reporting format.

Controllers

A controller 130 of system 100 of the present invention directs dipping of capillary elements into, for example, multiwell plate 150 to sample reagents such as enzymes and substrates, fluid recirculation baths or troughs, or the like. A variety of controlling instrumentation is also optionally utilized in conjunction with the microfluidic devices and handling systems described herein, for controlling the transport, concentration, direction, and motion of fluids and/or separation of materials within the devices of the present invention, e.g., by pressure-based control.

As described above, in many cases, fluid transport, concentration, and direction are controlled in whole or in part, using pressure based flow systems that incorporate external or internal pressure sources to drive fluid flow. Internal sources include microfabricated pumps, e.g., diaphragm pumps, thermal pumps, and the like that have been described in the art. See, e.g., U.S. Pat. Nos. 5,271,724, 5,277,556, and 5,375,979 and Published PCT Application Nos. WO 94/05414 and WO 97/02357. Preferably, external pressure sources are used, and applied to ports at channel termini. These applied pressures, or vacuums, generate pressure differentials across the lengths of channels to drive fluid flow through them. In the interconnected channel networks described herein, differential flow rates on volumes are optionally accomplished by applying different pressures or vacuums at multiple ports, or preferably, by applying a single vacuum at a common waste port (see, FIG. 1) and configuring the various channels with appropriate resistance to yield desired flow rates. Example systems are also described in U.S. Ser. No. 09/238,467 filed Jan. 28, 1999.

Typically, the controller systems are appropriately configured to receive or interface with a microfluidic device or system element as described herein. For example, the controller and/or detector, optionally includes a stage upon which the device of the invention is mounted to facilitate appropriate interfacing between the controller and/or detector and the device. Typically, the stage includes an appropriate mounting/alignment structural element, such as a nesting well, alignment pins and/or holes, asymmetric edge structures (to facilitate proper device alignment), and the like. Many such configurations are described in the references cited herein.

The controlling instrumentation discussed above is also used to provide for electrokinetic injection or withdrawal of material downstream of the region of interest to control an upstream flow rate. The same instrumentation and techniques described above are also utilized to inject a fluid into a downstream port to function as a flow control element.

Detector

The devices described herein include signal detectors 122 which detect fluorescence. In other devices, signal detectors may detect concentration, phosphorescence, radioactivity, pH, charge, absorbance, refractive index, luminescence, temperature, magnetism, mass (e.g., mass spectrometry), or the like. The detector(s) optionally monitors one or a plurality of signals from upstream and/or downstream (e.g., in or proximal to the separation region) of an assay mixing point in which, e.g., a ligand and an enzyme are mixed. For example, the detector optionally monitors a plurality of optical signals which correspond in position to “real time” assay/separation results.

Example detectors or sensors include photomultiplier tubes, CCD arrays, optical sensors, temperature sensors, pressure sensors, pH sensors, conductivity sensors, mass sensors, scanning detectors, or the like. Materials which emit a detectable signal are optionally flowed past the detector, or, alternatively, the detector can move relative to the array to determine the position of an assay component (or, the detector can simultaneously monitor a number of spatial positions corresponding to channel regions, e.g., as in a CCD array). Each of these types of sensors is optionally readily incorporated into the microfluidic systems described herein. In these systems, such detectors are placed either within or adjacent to the microfluidic device or one or more channels, chambers or conduits of the device, such that the detector is within sensory communication with the device, channel, or chamber. The phrase “within sensory communication” of a particular region or element, as used herein, generally refers to the placement of the detector in a position such that the detector is capable of detecting the property of the microfluidic device, a portion of the microfluidic device, or the contents of a portion of the microfluidic device, for which that detector was intended. The detector optionally includes or is operably linked to a computer, e.g., which has software for converting detector signal information into assay result information (e.g., kinetic data of modulator activity), or the like. A microfluidic system optionally employs multiple different detection systems for monitoring the output of the system. Detection systems of the present invention are used to detect and monitor the materials in a particular channel region (or other reaction detection region).

The detector optionally exists as a separate unit, but is preferably integrated with the controller system, into a single instrument. Integration of these functions into a single unit facilitates connection of these instruments with the computer (described below), by permitting the use of few or a single communication port(s) for transmitting information between the controller, the detector, and the computer.

Computer

As noted above, the microfluidic devices and integrated systems of the present invention include a computer 120 operably connected to the controller. The computer typically includes an instruction set, e.g., for varying or selecting a rate or a mode of dipping capillary or pipettor elements into fluid materials, for sampling fluidic materials (e.g., enzymes, substrates, reactants, chromatographic materials, eluents, separation buffers, etc.), or the like. Additionally, either or both of the controller system and/or the detection system is/are optionally coupled to an appropriately programmed processor or computer which functions to instruct the operation of these instruments in accordance with preprogrammed or user input instructions, receive data and information from these instruments, and interpret, manipulate and report this information to the user. As such, the computer is typically appropriately coupled to one or both of these instruments (e.g., including an analog to digital or digital to analog converter as needed).

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation, e.g., varying or selecting the rate or mode of fluid and/or microfluidic device movement, controlling flow rates within microscale channels, directing xyz translation of the microfluidic device or of one or more microwell plates, or the like. The computer then receives the data from the one or more sensors/detectors included within the system, and interprets the data, either provides it in a user understood format, or uses that data to initiate further controller instructions, in accordance with the programming, e.g., such as in monitoring and control of flow rates, temperatures, applied voltages, and the like. Additionally, the software is optionally used to control, e.g., pressure or electrokinetic modulated injection or withdrawal of material.

### Example 2

In vitro screening of kinase activity typically involves the use of a short peptide sequence used as an in vitro surrogate for the physiologically relevant protein substrate. An alternative embodiment of the invention may be employed to identify such substrate surrogates. Substrate plates for this purpose are arranged based on considerations similar to those taken into account for substrate plates that are matched with an enzyme plate as described above. I.e., the substrates are arranged based on factors such as, for example, electrophoretic mobility.

In an example assay, a kinase target of interest is incubated in a micro titer plate with a library of fluorescently labeled peptide substrate-candidates. The reaction is terminated and the mixture sipped onto a microfluidic chip. The extent of phosphorylation of each fluorescently labeled peptide is then measured by a mobility shift assay in accordance with the invention.

A control solution that contains only the peptide without any enzyme is provided for each peptide substrate-candidate. Thus, each multiwell plate includes two solutions containing each peptide, one solution without enzyme and one solution with enzyme. The control solution contains peptide, ATP, and reaction buffer. The enzyme solution contains the same components plus the enzyme. Both the control and enzyme-containing solutions are incubated for a predetermined period of time. At the end of that time, the reaction in the well with the enzyme-containing solution is terminated by the addition of an amount of termination buffer, while the same amount of separation buffer is added to the well containing the control solution. Addition of the same amount of material to both wells ensures that the final concentration of the labeled peptide in both wells is identical.

This example assay is carried out in a microfluidic device containing multiple external capillaries or “sippers” that allow samples from multiple sources to be simultaneously introduced to be processed in parallel within the microfluidic device. Examples of such microfluidic devices are provided in U.S. Pat. No. 6,358,387, which is assigned to the assignee of this invention. The location of the control well on the multiwell plate is such that while the solution containing both the enzyme and a particular substrate candidate is sipped on one sipper of a sipper chip, the control solution containing that same substrate candidate is also sipped onto the same chip through another sipper. After the enzyme-containing and control solutions enter the microfluidic device, both solutions are subjected to a common set of separation conditions (i.e. pressure and voltages). The use of the control solution becomes most critical when there is a high level of substrate conversion in the reaction well, as only one peak is present and so comparison of arrival time of peaks in the channels containing the enzyme-containing and control solutions indicates the presence of product formation.

In general, to achieve a desired resolution during the separation of a given peptide substrate and the product resulting from the interaction between the substrate and an enzyme, an optimum set of pressures and separation voltages are used. This would mean that for a panel of 54 peptides, 54 unique separation conditions would be used. Even with the automation afforded by the use of a microfluidic device, it may be cumbersome to manually input the separation conditions required for each peptide. A compromise solution involves grouping peptides that have similar separation conditions and separating them under a common set of conditions. For example, a constant pressure could be applied during the assay of each peptide, while different voltages could be applied for each different group of peptides.

In many embodiments, the substrate-candidate peptides can be broadly grouped into two groups: product-first and substrate-first assays. In a product-first assay, the product peak of a product-substrate pair reaches the detector first. An operating pressure was designated for product- and substrate-first assays; in our specific case we chose −1 psi for product-first assays and −1.6 for substrate-first assays. Next, voltage drops were chosen that give a resolution of 1.2 at the operating pressure. Peptides were then grouped together into groups of 6 where the operating voltage drops were within 200V of each other. Peptides were grouped into groups of 6 because a 12-sipper chip could sip 6 enzyme-containing wells and 6 control wells simultaneously. (Note: if a 4 sipper chip were used, peptides could be grouped into groups of 2.) An operating voltage drop was then chosen for this group of 6 peptides by choosing highest voltage drop common to all 6 peptides; in this way each product-substrate pair had a resolution of 1.2 or greater and assay sensitivity was not compromised. In some cases a group of 6 peptides could not be made and so a group of fewer than six was made with the balance being buffer-only wells. So in the case of 54 peptides used in our library, 21 were chosen to be product-first, which translated into 4 groups of 6 peptides with 3 buffer only wells. All groups operated at the same pressure of −1 psi, but each group had an incrementally increasing voltage drop as designated within a multi-line voltage script. The timing of application of the successive voltage drops was determined such that the peptides from the previous group had passed the detection zone and the new peptides had not yet been sipped. This in turn, coupled with the separation conditions, determined the throughput of the assay. The remaining 33 peptides were grouped on a separate plate and run substrate-first at a common pressure of −1.6 psi, resulting in 6 groups of 6 peptides with 3 buffer-only wells. Choice of voltage and timing of application of voltage is identical to the product-first case.

The substrates are placed on a multiwell plate in DMSO in a concentrated format such that when the enzyme and reaction buffer is used, the final substrate concentration is close to the desired assay conditions, e.g., store 1 μl of 76 gM substrate and then add to 50 pl of reaction buffer for a final concentration of 1.5 μM. These plates can then be made in bulk and frozen for use at a later date.

Without knowledge of the enzymatic kinetics, the reactions are typically carried out with a multi-hour incubation (>12 hours) and with high enzyme and ATP concentrations.

With this assay format, 12 kinase enzymes were run to look for substrates. One enzyme was PICA and was used only as a test case to see if the method found our regular kinase target.

Of the other 11 kinases, we found new targets for 8 of them. These enzymes and their new substrates are listed in the following table.

| | [E] | [ATP] | | Literature Substrate | |
|---|---|---|---|---|---|
| Enzyme | (nM) | (μM) | Co-Factor | (% Conversion) | Hits |
| MET | 10 | 250 | MnCl₂ | KKKSPGEYVNIEFG (70%) | FL-EDPIYEFLPAKKK-CONH2 (97%) |
| | | | | | FL-MAEEEIYGEFFAKKK-CONH2 (93%) |
| | | | | | FL-KMAEEEEYFELVAKKK-CONH2 (90%) |
| | | | | | FL-EAIYAAPFAKKK-CONH2 (88%) |
| | | | | | FL-EEEEYFFIIAKKK-CONH2 (86%) |
| | | | | | FL-KMAEEEEYVFIEAKKK-CONH2 (85%) |
| | | | | | FL-KEDPDYEWPSAK-CONH2(63%) |
| | | | | | FL-MAAEEEYFFLFAKKK-CONH2 (62%) |
| | | | | | FL-EGIYGVLFKKK-CONH2 (60%) |
| MST2 | 10 | 100 | MnCl₂ | MBP (N/A) | FL-KKSRGDYMTMQIG-CONH2 (75%) |
| | | | | | FL-ENDYINASLKKK-CONH2 (60%) |
| | | | | | FL-PLARTLSVAGLPGKK-COOH(40%) |
| IKKα | 3.5 | 100 | MnCl₂ | IKK-tide (N/A) | FL-AKRRRLSSLRA-COOH (43%) |
| IGF-1R | 10 | 250 | MnCl₂ | KKKSPGEYVNIEFG (30%) | FL-KKSRGDYMTMQIG-CONH2 (55%) |
| | | | | | FL-MAAEEEYFFLFAKKK-CONH2 (60%) |
| FGFR3 | 5 | 250 | MnCl₂ | Poly-Glu Tyr (N/A) | FL-EDPIYEFLPAKKK-CONH2 (100%) |
| | | | | | FL-KMAEEEEYFELVAKKK-CONH2 (100%) |
| | | | | | FL-KMAEEEEYFELVAKKK-CONH2 (100%) |
| | | | | | FL-KMAEEEEYVFIEAKKK-CONH2 (100%) |
| | | | | | FL-MAAEEEYMMMMAKKK-CONH2 (78%) |
| | | | | | FL-KKKSPGEYVNIEFG-CONH2 (73%) |
| EGFR | 5 | 250 | MnCl₂ | Angiotensin II (N/A) | FL-EDPIYEFLPAKKK-CONH2 (98%) |
| | | | | | FL-KMAEEEEYFELVAKKK-CONH2(90%) |
| | | | | | FL-MAEEEIYGEFFAKKK-CONH2 (87%) |
| | | | | | FL-KKKSPGEYVNIEFG-CONH2 (50%) |
| | | | | | FL-KEDPDYEWPSAK-CONH2 (50%) |
| | | | | | FL-EAIYAAPFAKKK-CONH2 (46%) |
| | | | | | FL-ENDYINASLKKK-CONH2(44%) |
| IKKβ | | | MnCl₂ | IKK-tide (N/A) | FL-AKRRRLSSLRA-COOH (74%) |
| ERK2 | | | | | FL-IPTSPITTTYFFFKKK-COOH (100%) |
| (MAPK2) | | | | | |
| | | | | | FL-APRTPGGRR-COOH (60%) |
| MEK1 | 7 | 100 | MnCl₂ | N/A | No Hit |
| JNK2a | 60 | 100 | MnCl₂ | N/A | No Hit |
| MKK6 | | | | | No Hit |

This table lists the substrates as hits with the peptide sequence and the 90 conversion in parentheses. Where possible, data is shown for the enzyme with a known peptide sequence for the enzyme. In addition, assay conditions are listed. In all cases, the enzyme and substrate were incubated overnight (i.e., ˜12-16 hours).

While the foregoing invention has been described in some detail for purposes of clarity and understanding, it will be clear from a reading of this disclosure that various changes in form and detail can be made without departing from the true scope of the invention. For example, all the techniques and apparatus described above may be used in various combinations.

All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A kit for screening a compound for enzyme inhibition activity, the kit comprising:
a first multiwell plate having a plurality of enzymes disposed within a first plurality of wells;
a second multiwell plate having a plurality of enzyme substrates disposed within a second plurality of wells;
a phosphate source disposed within each well of the second plate, the phosphate source disposed in the well at a predetermined concentration; and
a cofactor disposed within each well of the second plate, the cofactor disposed in each well at a predetermined concentration.

2. The kit of claim 1 wherein the first plurality of wells of the first multiwell plate are disposed evenly upon the plate in 24 columns, each column including a different enzyme disposed within the wells of the column.

3. The kit of claim 2 wherein the order in which the enzymes are disposed on the first plate is based on similarity of electrophoretic mobility.

4. The kit of claim 2 wherein the order in which the enzymes are disposed on the first plate from column 1 to column 24 is MAPKAPK2, AurA, PKCζ, RSK1, MAPKAPK5, Erk1, PKD2, CK1δ, CHK1, ABL, FYN, LYNa, CHK2, MET, LCK, SRC, GSK3β, Erk2, PKACα, AKT2, INSR, p38α, AKT1, and MSK1.

5. The kit of claim 2 wherein the order in which the enzymes are disposed on the first plate from column 1 to column 24 is PKCβ2, ROCK2, CDK2, MST2, PKG1α, PAK2, IGF1R, FGFR1, MARK1, CAMK2δ, PIM2, BTK, c-TAK1, DYRK1a, CaMK4, AMPKα1, FLT3, HGK, KDR, Raf-1, P70S6K, IRAK4, SGK, and SYK.

6. The kit of claim 1 further comprising a reconstitution buffer.

7. The kit of claim 1 further comprising a termination buffer.

8. A system for screening a compound for enzyme inhibition activity, the system comprising:
a kit including first and second multiwell plates, the first plate having a plurality of enzymes disposed within a first plurality of wells, the second plate having a plurality of enzyme substrates disposed within a second plurality of wells, wherein a phosphate source is disposed at a predetermined concentration within each well of the second plate, and wherein a cofactor is disposed at a predetermined concentration within each well of the second plate; and
a microfluidic device, the microfluidic device having at least one microchannel and a capillary element, the capillary element operably connected to and in fluid communication with the microchannel.

9. The system of claim 8 further comprising a detector operably connected to the microfluidic device and a computer.

10. The system of claim 9 further comprising a controller operably connected to the computer.

11. The system of claim 10 further comprising a fluid direction system operably connected to the computer, the fluid direction system including a pressure source in fluid communication with the microfluidic device.
